(19) United States
(12) Patent Application Publication
(10) Pub. No.: US 2024/0408306 A1
(43) Pub. Date: Dec. 12, 2024

(54) MODULATED POWER INJECTOR WITH INPUT DEVICE

(71) Applicant: Osprey Medical, Inc., Minnetonka, MN (US)

(72) Inventors: Rodney L. Houfburg, Prior Lake, MN (US); Dale Brady, New Brighton, MN (US); Matthew M. Burns, Orono, MN (US); Steve Rathjen, South Lake Tahoe, CA (US); Alexander Frederick Dietz, Minneapolis, MN (US)

(73) Assignee: Osprey Medical, Inc., Minnetonka, MN (US)

(21) Appl. No.: 18/417,077

(22) Filed: Jan. 19, 2024

Related U.S. Application Data
(63) Continuation of application No. 17/471,961, filed on Sep. 10, 2021, now abandoned.
Application No. 17/471,961 is a continuation-in-part of application No. 16/931,664, filed on Jul. 17, 2020, now abandoned.
(60) Provisional application No. 63/166,679, filed on Mar. 26, 2021.
(60) Provisional application No. 62/875,859, filed on Jul. 18, 2019.

(51) Int. Cl. A61M 5/20; A61M 5/315

(52) U.S. Cl. CPC A61M 5/20; A61M 5/31528; A61M 5/31578; A61M 2005/2026; A61M 2205/3327; A61M 2205/3334; A61M 2205/3553; A61M 2205/52; A61M 2205/8206

(57) ABSTRACT

A system for injecting medium into a patient includes an automated injector including a reservoir, an ejector for ejecting a volume of fluid medium from the reservoir, and an actuator coupled to the ejector. An input device includes a syringe housing, a plunger, a circuit board coupled to a first component of the input device, a plunger position sensor, a battery, and a transmitter for sending an input device action signal to the automated injector. The input device action signal is based at least in part on a signal sent from plunger position sensor. A diversion apparatus is disposed downstream from the reservoir and is configured to receive at least a first portion of the volume of the fluid medium ejected from the reservoir.

### CROSS-REFERENCE TO RELATED APPLICATIONS

This application is a continuation of U.S. patent application Ser. No. 17/471,961, filed Sep. 10, 2021, which claims priority to and the benefit of U.S. Provisional Patent Application No. 63/166,679, filed Mar. 26, 2021, entitled “MODULATED POWER INJECTOR WITH INPUT SYRINGE”, the entire disclosures of which are incorporated herein by reference in their entireties. This application is also a continuation-in-part of U.S. patent application Ser. No. 16/931,664, filed Jul. 17, 2020, entitled “SYSTEMS AND METHODS FOR MEASURING INJECTED FLUIDS”, which application claims the benefit of priority to U.S. Provisional Patent Application No. 62/875,859, filed Jul. 18, 2019, the entire disclosures of which are incorporated herein by reference in their entireties.

### BACKGROUND

Powered injectors may be used to inject medicine, saline, contrast, or other medicaments or fluids into a patient undergoing a medical procedure. Automated injectors are typically controlled by pressing a control button thereon.

### SUMMARY

In one aspect, the technology relates a system for injecting medium into a patient, the system including: an automated injector including: a medium reservoir; an ejector for ejecting a volume of a fluid medium from the medium reservoir; and an actuator coupled to the ejector; an input device remote from and communicatively coupled to the actuator, wherein the input device includes: a syringe housing; a plunger slidably received in the syringe housing; a circuit board coupled to a first component of the input device; a plunger position sensor; a battery coupled to the circuit board and configured to provide power to the plunger position sensor; and a transmitter coupled to the circuit board for sending an input device action signal to the automated injector, wherein the input device action signal is based at least in part on a signal sent from plunger position sensor; and a diversion apparatus disposed downstream from the reservoir, wherein the diversion apparatus is configured to receive at least a first portion of the volume of the fluid medium ejected from the medium reservoir. In an example, the transmitter includes a wireless transmitter and wherein the automated injector includes a wireless receiver for receiving the input device action signal. In another example, the input device further includes a spring for biasing the plunger relative to the syringe housing. In yet another example, the medium reservoir includes a syringe barrel; the ejector includes a plunger slidably disposed in the syringe barrel; and the actuator includes a lead screw and a motor coupled to the lead screw, wherein a rotation of the lead screw advances the ejector within the syringe barrel. In still another example, the automated injector further includes a position sensor for detecting a position of at least one of the ejector and the lead screw.

In another example of the above aspect, the system further includes a patient connection element downstream of the diversion apparatus for receiving at least a second portion of the volume of the fluid medium ejected from the medium reservoir. In an example, the first portion of the volume of the fluid medium and the second portion of the volume of the fluid medium includes the volume of the fluid medium ejected from the medium reservoir. In another example, the diversion apparatus includes a waste vessel for receiving at least a portion of the first portion of the volume of the fluid medium. In yet another example, the plunger position sensor includes at least one Hall Effect sensor coupled to the first component and a magnet coupled to a second component of the input device, wherein the first component is moveable relative to the second component. In still another example, the plunger position sensor includes at least one of a light emitter, a light receiver, a potentiometer, and a magnet.

In another aspect, the technology relates to a system for injecting medium into a patient, the system including: an automated injector including: a medium reservoir; an ejector for ejecting a volume of a fluid medium from the medium reservoir; and an actuator coupled to the ejector; an injection sensor disposed proximate an outlet of the medium reservoir; an input device remote from and communicatively coupled to the actuator, wherein the input device includes: a syringe housing; a plunger slidably received in the syringe housing; a circuit board coupled to a first component of the input device; a plunger position sensor; a battery coupled to the circuit board and configured to provide power to the plunger position sensor; and a transmitter coupled to the circuit board for sending an input device action signal to the automated injector, wherein the input device action signal is based at least in part on a signal sent from plunger position sensor; a processor; and memory storing instructions that, when executed by the processor, cause the automated injector to perform operations including: controlling the actuator so as to advance the ejector at a first rate based at least in part on the input device action signal; and controlling the actuator so as to advance the ejector at a second rate different than the first rate based at least in part on an injection pressure signal received from the injection sensor. In an example, the processor and the memory are disposed on the automated injector. In another example, the position sensor includes at least one of a Hall Effect sensor, light emitter, a light receiver, a potentiometer, and a magnet. In yet another example, controlling the actuator so as to advance the ejector at a second rate includes: determining a target flow rate of the fluid medium proximate the injection sensor; and maintaining the target flow rate for a predetermined time, wherein the predetermined time is measured from the time that the target flow rate was determined or a variable time as a function of an input device. In still another example, the target flow rate is determined based at least in part on the injection pressure signal sent from the injection sensor.

In another example of the above aspect, the target flow rate is determined based at least in part on a signal sent from a flow sensor.

In another aspect, the technology relates to a method of controlling injection of a medium into a patient with an automated injector, the method including: receiving an input device action signal from an input device located remote from the automated injector; processing the input device action signal to obtain a first actuation signal; sending the first actuation signal, wherein the first actuation signal activates an actuator to eject the medium from the automated injector at a first rate; receiving a modification signal from at least one of the input device and a sensor; processing the modification signal to obtain a second actuation signal; and sending the second actuation signal based at least in part on the modification signal. In an example, the sensor is disposed remote from the automated injector. In another example, the sensor senses a pressure within a medium delivery system fluidically coupled to the automated injector and the patient. In yet another example, the sensor is disposed within the automated injector and senses a pressure of medium within a medium reservoir of the automated injector.

### BRIEF DESCRIPTION OF THE DRAWINGS

FIG. 1 depicts a power injector system, including an automated power injector that may be actuated by a hand-held input device.

FIG. 2A depicts a perspective view of an embodiment of a hand-held input device utilizing a measurement sensor module.

FIG. 2B depicts a partial perspective sectional view of the input device of FIG. 2A, depicting the measurement sensor module comprising a Hall Effect sensor module.

FIG. 2C depicts a partial exploded perspective view of the input device of FIG. 2B.

FIG. 3 depicts a perspective view of another example of an input device utilizing a Hall Effect sensor module.

FIG. 4 depicts a system utilizing an automated power injector having a screw-drive motor mechanism to receive, process, and activate a motor to drive a piston and plunger, as inputted by a hand-held input device.

FIG. 5 depicts a system having an automated power injector, a diversion apparatus, a collection reservoir and sensors to measure an injected medium from an injector and the medium diverted to the collection reservoir.

FIG. 6 presents some graphical results from an abstract titled: Comparison of Contrast Injection Pressure Contours with Different Methods for Coronary Angiography, SCAI 2020 Scientific Sessions, May 14-16, 2020, the disclosure of which is hereby incorporated by reference herein in its entirety.

FIG. 7 depicts graphically an injection profile of a medium delivered to a patient, identifying “under-injection” and “over-injection” volume areas of media for opacification purposes.

FIG. 7A depicts graphically a pulsatile medium injection profile of medium into a patient.

FIGS. 8A-10 depict example injection profiles, as delivered to a patient.

FIG. 11 depicts one example of a suitable operating environment in which one or more of the present examples may be implemented.

FIG. 12 depicts a method of controlling ejection of a medium from an automated injector.

FIG. 13 depicts a method of controlling ejection of a medium from an automated injector.

### DETAILED DESCRIPTION

This disclosure pertains to systems, devices, and methods used to control, transform or otherwise modulate the delivery of a substance, such as radiopaque contrast, to a delivery site and/or systems, devices, and methods that may be used to measure or otherwise make quantitative assessments of a medium delivered to a delivery site. More specifically, it is the intention of the following systems, devices, and methods to modulate and/or assess the delivery of media to a vessel, vascular bed, organ, and/or other corporeal structures so as to optimize the delivery of media to the intended site, while reducing inadvertent or excessive introduction of the media to other vessels, vascular beds, organs, and/or other structures, including systemic introduction.

The terms medium (media), agent, substance, material, medicament, and the like, are used generically herein to describe a variety of fluidal materials that may include, at least in part, a substance used in the performance of a diagnostic, therapeutic and/or/prophylactic medical procedure and such use is not intended to be limiting.

Some of the systems, devices and methods described herein may be used in conjunction with injection systems that may be automated with respect to the input, including devices and methods so as to optimize the delivery of a media to the intended site, while reducing inadvertent and/or excessive introduction of the media.

The technologies described herein are related to those presented in US 2021/0018348 (SYSTEMS AND METHODS FOR MEASURING INJECTED FLUIDS), the disclosure of which is hereby incorporated by reference herein in its entirety. In US 2021/0018348, systems, devices and methods have been disclosed to modulate and/or alter an injection from an automated injector, wherein a medium injected by the injector may be subjected to a diversion pathway placed in fluid coupling with the injector and a catheter utilized to deliver the medium to a patient's injection site within the body. The diversion pathway acts to divert a portion of the injection to the patient, so as to optimize the injection for visualization (i.e., angiography) but reduce needless contrast injected corporeally.

There are numerous occasions in the diagnostic, prophylactic and treatment practice of medicine wherein an agent, medicant, or medium is preferably delivered to a specific site within the body, as opposed to a more general, systemic introduction. One such exemplary occasion is the delivery of contrast media to coronary vasculature in the diagnosis (i.e., angiography) and treatment (e.g., balloon angioplasty and stenting) of coronary vascular disease. The description, as well as the devices and methods described herein, may be used in modulating (or otherwise altering, or regulating) and/or monitoring/measuring medium delivery to the coronary vasculature in prevention of toxic systemic effects of such an agent. One skilled in the art, however, would recognize that there are many other applications wherein the controlled delivery and/or quantitative assessment of a media to a specific vessel, structure, organ or site of the body may also benefit from the devices and methods disclosed herein. For simplicity, these devices and methods may be described as they relate to contrast media delivery modulation and/or measurement. As such, they may be used in the prevention of Contrast Induced Nephropathy; however, it is not intended, nor should it be construed, so as to limit the use to this sole purpose. Exemplary other uses may include the delivery, injection, modulation, or measurement of: cancer treatment agent to a tumor, thrombolytic to an occluded artery, occluding or sclerosing agent to a vascular malformation or diseased tissue; genetic agent to a muscular bed, neural cavity or organ, emulsion to the eye, bulking agent to musculature and/or sphincter, imaging agent to the lymphatic system, antibiotics to an infected tissue, supplements in the dialysis of the kidney, to name but a few.

There are many different types of automated power devices or automated power injectors (APIs) for injecting a medium into a patient. These devices may be used in lieu of injecting a medium by a hand-held syringe. APIs may be defined by their use and the type of medium they may automatically inject, for example an MRI, CT, or angiography injector. Each type of API may have different use requirements and deliver different mediums by the apparatus. Differing agents injected, and the site the medium agent may be delivered to, may be vastly different with respect to access type (where in the body a catheter or needle might enter the body), the site at which the medium is to be delivered, and the conditions of the delivery apparatus that might be required (i.e., conduit size, pressure to deliver, etc.) These are but a few of the considerations in the use of the various power injectors. Given the requirements of the medicant/medium and delivery, automated power injectors may have differing mechanisms to drive the fluid medium, such as: piston or plunger pumps, diaphragm pump, gear pump, centrifugal pump, hydraulic pump, gear pump, screw pump, to name a few.

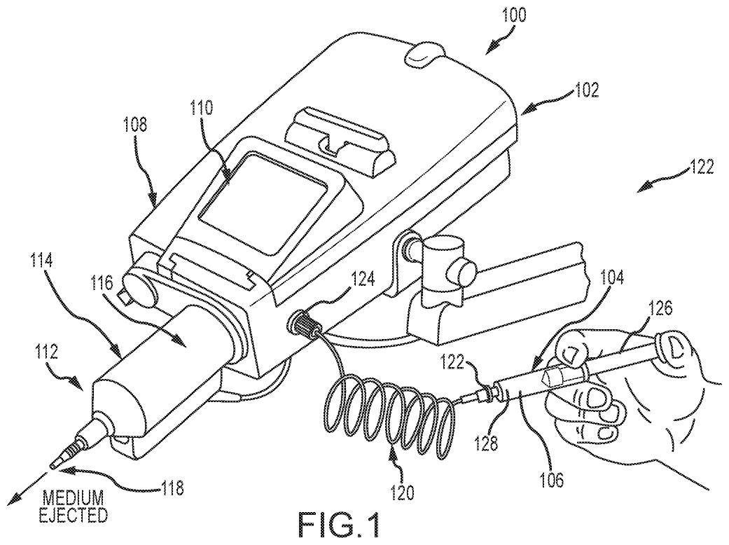

FIG. 1 depicts a power injector system 100, including an automated power injector (API) 102 that may be actuated by a hand-held input device 104. The API 102 is depicted for the power injection of a medium that may be used for injecting a medium during angiography, for example. As can be seen, the injector 104 is in the form of a hand-held syringe body 106 that controls the advancement/injection of a medium into a patient. A number of components disposed within the depicted elements are not shown, but are shown in other figures in the present application, or would otherwise be apparent to a person of skill in the art.

The API 102 of FIG. 1 may include a housing 108 to house an internal drive motor (not shown) and a data display 110 (indicating status/operating parameters of the API 102). An API syringe 112, including an API barrel 114 and plunger 116, may be mounted on the API 102 so as to interface with an internal drive mechanism/motor (not shown). The plunger 116 may be connected to a piston that is further coupled to a motor drive screw (not shown), for example, whereby the motor may cause the API plunger 116 to move along the API barrel 114 to eject contents of the syringe 112 (or to draw fluids into) through the barrel exit/outlet 118.

FIG. 1 depicts an input device 104 configured as a hand-held syringe 106 and a conduit 120 connecting an outlet 122 of the hand-held syringe 106 to the API 102, typically at a port 124 on the housing 108. Input signals received from the syringe 106 may be delivered to a processor/circuit board (not shown), and the processor/board may then direct/control an actuator (not shown) in the API 102 to control the movement of a motor drive screw (not shown), so as to drive the piston/plunger 116 into the API barrel 114.

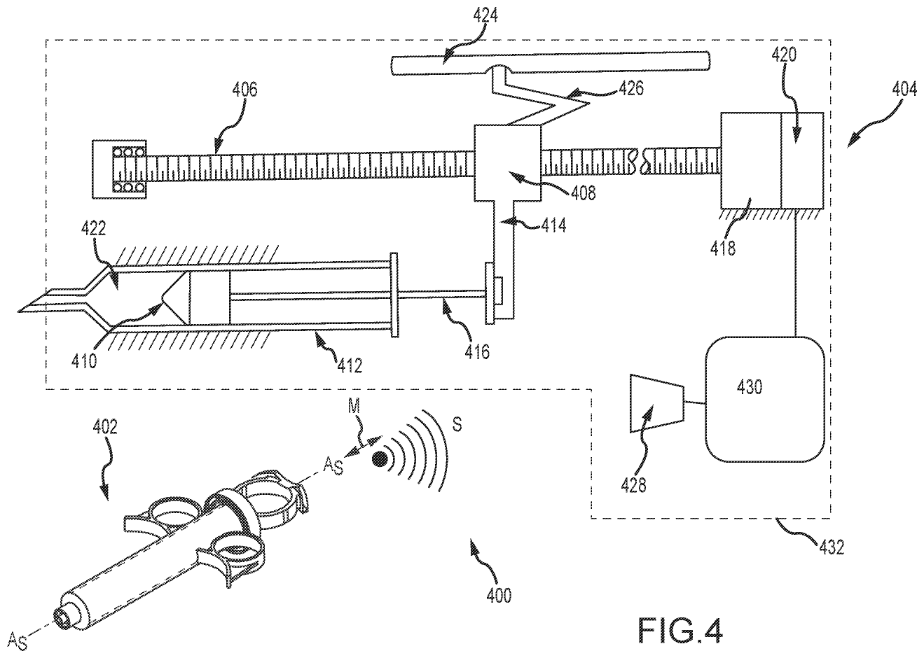

In the input device 104 of FIG. 1, the position of the plunger 126 relative to the input barrel 128 may be detected. Structures utilized for this detection include, but are not limited to, sensing systems that may include one or more magnets and potentiometers, light sensors, and Hall Effect sensors (to name a few). Such devices with detection functionality may be advantageously used as the input device 104 depicted in FIG. 1 to provide input to the API 102. The input device 104 may be configured to mimic (as to feel and performance) hand-held injection syringes that are often used for the direct injection of contrast. In this case, the input devices 104 (as further described herein) may be readily accepted by surgeons and other providers who may prefer the performance advantages available with an API 102, but who may have more experience with the look and feel of a hand-held injection device. Another advantage of an untethered (i.e., wireless) hand-held input devices (such as, for example, 402 of FIG. 4) is that they may be used in a sterile field without being tethered to the API 102, since they can communicate with the API 102 through Bluetooth, IR, RF, Wi-Fi, or other wireless communication protocols and devices (e.g., in lieu of being tethered by the conduit 120, as depicted in FIG. 1). These hand-held input devices 104 may be disposable as well, removing the need to re-sterilize with different patients. These are but a few of the advantages of using a hand-held input device 104 as the input to an API 102.

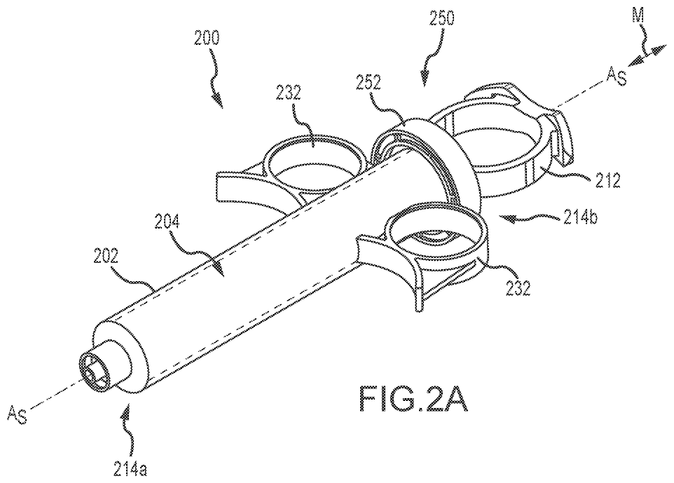

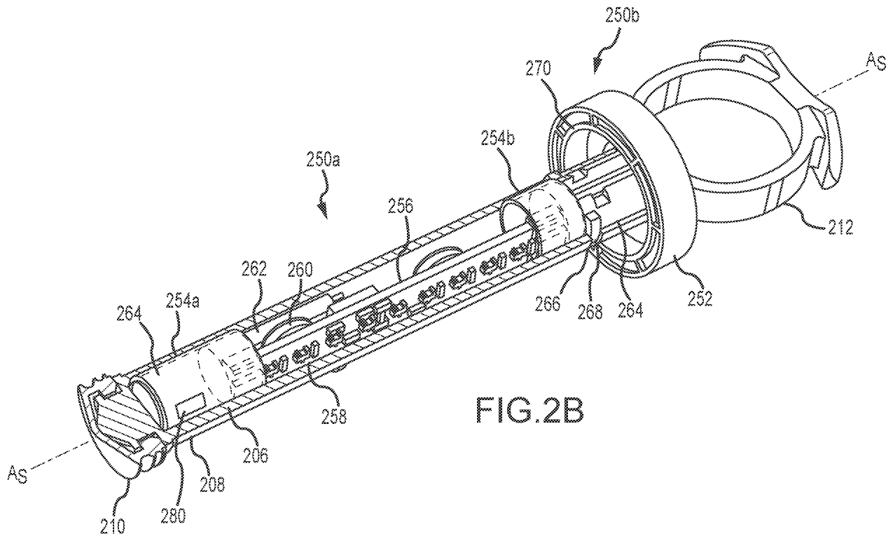

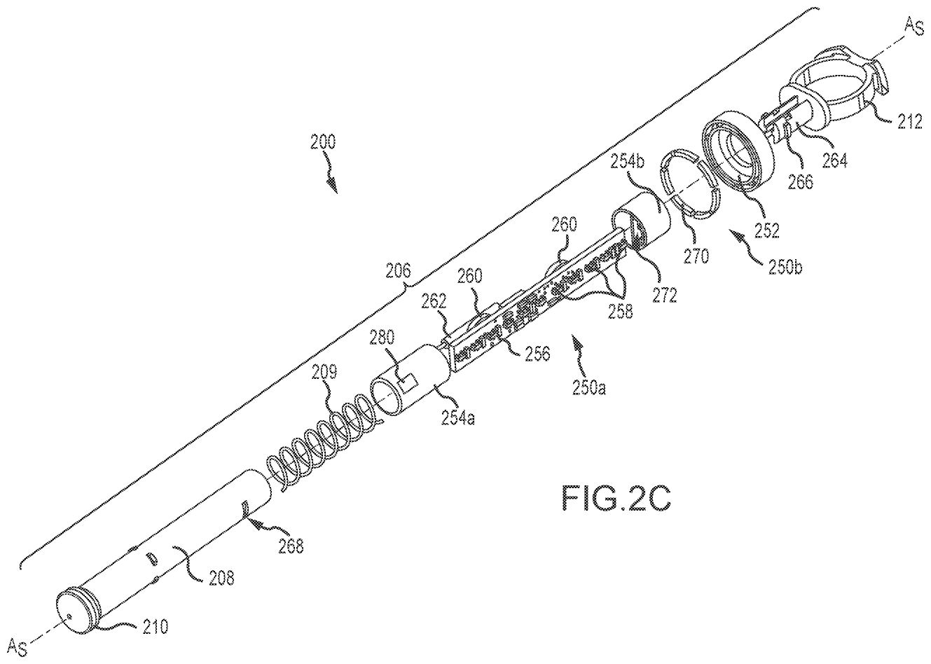

FIG. 2A depicts a perspective view of an embodiment of a hand-held, syringe-type input device 200 utilizing a Hall Effect sensor module, which is described in more detail below. The input device 200 is configured to appear and function in a manner similar to known syringes, with a similar look and feel during use. The device 200 includes a syringe housing 202 defining an inner bore 204. A plunger or piston, which is described in more detail below, is slidably received in the bore 204. More specifically, the piston is slidably engaged with an interior surface of the bore 204 and linear movement M of a plunger shaft within the bore 204 moves the piston. Movement M is along the syringe axis As. A thumb ring (or palm plunger, or similar attachment element) 212 may be utilized to push and pull the plunger along axis As, as described in more detail below. The discharge end 214a may be occluded and/or sealed for the syringe to act as a virtual input, rather than to actually deliver a fluid medium out of its discharge end 214a. Alternatively, a spring or similar elastic element may be positioned within the bore, between the plunger 210 (FIG. 2B) and the discharge end 214a so as to provide tactile feed back to the physician. As the plunger is moved M in a direction towards the discharge end 214 of the syringe housing 202, wireless/wired signals may be transmitted to a receiver (e.g., as depicted in FIG. 4), and the receiver may input information to a processor/circuit board (FIG. 4). The processor could be integral with the API, or it could be a standalone device. In one aspect, the data signals may be transmitted wirelessly (Bluetooth Low Energy, IR, RF, etc.). Further, the processor may interpret/process data received and send signals to an actuator to control/drive/actuate a motor to drive a screw-drive, for example, to drive a piston/plunger of the API. The data received by the hand-held input device may be representative of the actual volume, and rate of injection, by the virtual hand-held device. In another aspect, the data may simply be an “off/on” switch, initiating API discharge when a signal is received, and ceasing discharge when the hand-held syringe stops motion or is pulled back, out of the barrel. In either case, data sent by the input syringe to the API, or an internal data collection related to the discharge from the API barrel/plunger, can be utilized to ensure the volume of fluid ejected from the API.

Further in the description of FIG. 2A, two finger rings or tabs 232 may receive the fingers of a user during use. Note that throughout the description a cylindrical-type housing 202 and inner bore 204 are described; however, it is contemplated that there may be a variety of constructions of a housing/bore 202/204 and piston/plunger 206/210 that may provide the function as anticipated herein and the shape (including rectangular, ovular, triangular cross-section, etc.), in and of itself, should not be limiting. The input syringe 200 also includes a Hall Effect sensor module 250, described in more detail below. One component of the Hall Effect sensor module 250 may be a magnet retention ring 252, which is disposed on an outer or exterior surface of the syringe housing 202. In the depicted embodiment, the magnetic retention ring 252 is disposed proximate a proximal end 214b of the housing 202, but it may be disposed in other locations along the housing 202.

FIG. 2B depicts a partial perspective sectional view of the input device 200 of FIG. 2A, depicting the Hall Effect sensor module 250 (comprising, as an example, 250a and 250b). Certain components 250a of the Hall Effect sensor module 250 may be disposed within an inner chamber of a hollow shaft 208 of the plunger 206, while certain components 250b may be disposed on an exterior surface of the syringe housing. These various components 250a, 250b are described in more detail below. So-called internal components 250a (i.e., internal to the plunger 206) may include retention inserts 254a, 254b, a base or circuit board 256, and a single or plurality of Hall Effect sensors 258 disposed thereon. One or more batteries 260 and a control switch 262 may also be secured to the circuit board 256. Signals from the Hall Effect sensor(s) 258 may be first processed by the circuit board 256, which may determine the position of the plunger 206, the volume of media in the syringe, etc., and then send this information to an API receiver (or other associated processing system) via the transmitter 280. In another embodiment, e.g., if a non-processing base 256 is used, the signals from each Hall Effect sensor 258 may be sent directly via the transmitter 280 to an alternative associated system for processing data/signal(s).

The distal retention insert 254a may be inserted into the shaft 208 so as to be near the piston 210. The distal retention insert 254a may define a void 264, which may contain a wireless transmitter 280, such as a Bluetooth transmitter. The transmitter 280 may send signals from the Hall Effect sensors 258 to an associated signal processing device such as described herein. In an alternative embodiment, a cable connection such as described above, may be utilized. The proximal retention insert 254b is disposed in the hollow shaft 208 near the thumb ring 212. Together, the distal retention insert 254a and the proximal retention insert 254b support, protect, and retain the circuit board 256 within the hollow shaft 208. These two components may be configured for a snug fit in the shaft 208, or may include a key or other projection to engage with an opening or slot in the shaft 208, so as to prevent rotation of the board 256 within the shaft 208. The retention inserts 254a, 254b may be permanently fixed within the shaft 208, although configuring the inserts 254a, 254b for removal may be advantageous so as to allow for replacement or repair of the circuit board 256, batteries 260, etc. In one embodiment, the thumb ring 212 may include a resilient base 264 including a plurality of projections 266 that may be engageable with mating slots 268 in the shaft 208. Disengaging these projections 266 allows for removal of the retention inserts 254a, 254b and other internal components. A plurality of Hall Effect sensors 258 are depicted. A greater or fewer number of sensors 258 may be utilized in various embodiments, although a greater number of sensors 258 may provide for more accurate determinations with regard the position of the plunger 206 (and thus, the speed and volume of the sensed input syringe). The Hall Effect sensors 258 are disposed linearly within the chamber so as to be substantially aligned with, or parallel to, the axis AS. Although the previous description includes various components of the input syringe and their proximity to one another, it is clear that these associations are only exemplary and other configurations may be utilized to obtain the same outcome, including a construction wherein the Hall Effect sensors are mounted on the housing and the magnet component is fixed to the plunger.

In further describing the embodiment of Hall Effect sensor(s) attached to the shaft and magnet(s) attached to the housing, external components 250b may include the magnet retention ring 252, which may hold a plurality of magnets 270, such as arc magnets, in the depicted embodiment. In other embodiments, cube, cylindrical, or other magnets may be utilized. The positions of the magnets 270 are fixed relative to, and about, the input syringe housing. The arc magnets 270 form a substantially circular magnetic field through which the shaft 208 (and the Hall Effect sensors 258) pass when the shaft 208 is withdrawn from, or inserted into, the inner bore of the syringe. The circular magnetic field enables the Hall Effect sensors 258 to detect the field, regardless of the rotational position of the plunger 206 about the axis AS. In other embodiments, the magnets 270 may be secured directly to the syringe housing without the magnet retention ring.

FIG. 2C depicts a partial exploded perspective view of a portion of the hand-held input syringe 200, as seen in FIG. 2B. More specifically, the plunger 206, Hall Effect sensor module internal components 250a, and Hall Effect sensor module external components 250b are depicted. In general, certain of these components are described above in FIGS. 2A-2C and are not necessarily described further. In the depicted embodiment, however, both the distal retention insert 254a and proximal retention insert 254b include shaped recesses 272 that may be configured to receive the circuit board 256 so as to hold that element in place. The recesses 272 may be disposed in the inserts 254a, 254b so as to conserve space within the hollow shaft 208 of the plunger 206. On a side of the circuit board 256 (in the case that the board may due some processing of the Hall Effect sensor signals), opposite the Hall Effect sensors 258, may be disposed one or more batteries 260. Additionally, a switch 262 may be disposed proximate the batteries 260, or elsewhere within the hollow shaft 208. The switch 262, in certain embodiments, may be a reed switch that detects plunger movement and moves to an engaged or activated position. The switch 262 is not required but may help preserve power when the syringe 200 is not in use. When activated, the switch 262 may selectively connect power from the one or more batteries 260 to either or both of the plurality of Hall Effect sensors 258, as well as the wireless transmitter 280. In other embodiments, a manually-operated switched, such as a pull tab, button, or rocker switch may be actuated by the user. In other examples, a single Hall Effect sensor may be utilized instead of a plurality of sensors.

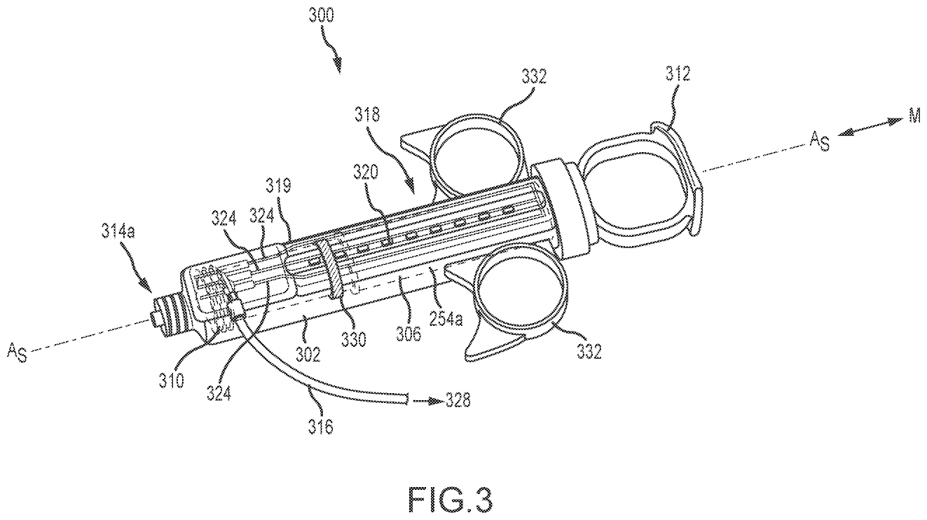

FIG. 3 depicts a perspective view of another embodiment of a hand-held input syringe 300 utilizing a Hall Effect sensor module. The input syringe 300 may include a syringe housing 302 defining a hollow inner bore. A plunger 306, including a shaft 308 and a piston 310, is slidably received in the bore. Even though fluid is not being discharged from the input syringe 300, the piston 310 is desirable to maintain stability of the shaft 308 as it is advanced within the syringe housing 302. More specifically, the piston 310 may be slidably engaged with an interior surface of the bore and linear movement M of the shaft 308, within the bore, moves the piston 310. Movement M is along the syringe axis As. The plunger 306 is moved back and forth within the bore 304 by the movement of a thumb pad, thumb-ring 312, or an alternative construction to provide movement of the plunger. As the plunger 306 is moved M in a direction towards the discharge end (opposite the thumb-ring 312) of the syringe housing 302, wireless (e.g., Bluetooth, Wi-Fi, IR, RF, etc.) signals may be sent by a transmitter to a receiver in communication with a processor associated with the movement and/or control of an API.

As an alternative embodiment to that depicted in FIGS. 2A-2C, a Hall Effect sensor module 318 may be secured to an exterior surface of the syringe housing 302, rather than securement to the plunger. The Hall Effect sensor module 318 includes a Hall Effect sensor housing 319 that encloses a plurality of Hall Effect sensors 320. As described above with regard to FIGS. 2A-2C, a greater number of discrete Hall Effect sensor elements may improve sensor accuracy. One or more leads or wires 324 may extend from an end of the Hall Effect sensor module 318. A cable 316 may connect at an end 328 to an API, such as depicted in FIG. 1. In other embodiments, communication may be via a radio, Bluetooth, or other wireless connection, as described herein. Information may include a rate of movement of the plunger 306 within the housing 302, thus providing information as to a total volume to be injected by the API in response to a movement of the plunger 306. As described above, the signals from the Hall Effect sensors may first be processed by an associated circuit board then sent to the API, or the discrete signals themselves may be sent to the API for processing.

In the depicted embodiment of FIG. 3, the shaft 308 of the plunger 306 has one or more magnets 330 disposed thereon or within the shaft 308. The magnet 330, in this case, may include a plurality of arc magnets disposed about the shaft 308. As the plunger 306 is moved in direction M along the axis As, the magnet 330 passes in front of the Hall Effect sensors 320 of the Hall Effect sensor module 318. The magnetic field generated by the magnet 330 is detected by the Hall Effect sensor 320. The Hall Effect sensor 320 sends a signal to the interface unit that determines the position of the plunger 306 within the syringe housing 302, based on the position of the magnet 330 as detected by an individual Hall Effect sensor 320. Thus, the position of the plunger 306 may be determined. The interface may also determine the various types of information listed above (as well as the rate of the plunger movement). Two finger rings or tabs 332 may be present to receive the fingers of a user during use. A stop may prevent the plunger 306 from being pulled out of the syringe housing 302.

Although the embodiments depicted in FIGS. 2A-3 depict a plurality of Hall Effect sensors, other embodiments of hand-held, input devices may utilize one or more sensors of various types. For example, a single sensor, or multiple sensors, may be used to measure a magnetic field, material resistance, capacitance, light transparency, etc. The measurements from such sensors may be utilized to determine the linear position, and movement rate, of a plunger within a syringe. Examples of such sensors include, but are not limited to, Hall Effect sensors (as described in more detail herein), inductive sensors, capacitive touch sensors, and others.

FIG. 4 depicts a system 400 utilizing a hand-held input device/syringe 402 to drive/control an API 404. In this example, a lead screw 406 may have a threaded engagement with a moveable screw drive element 408. As depicted, the screw drive element 408 may be affixed to the plunger 410 of an API syringe barrel 412 with a plunger driver 414. In this case, the plunger 410, barrel 412, and a plunger piston 416 may be off-set from the lead screw 406 and moveable screw drive element 408, only as an example. Furthermore, as discussed previously, there may be many modes/motors that may be used in injecting a medium by an automated power injector, and this is only but one example.

As further depicted in FIG. 4, a motor 418 and a motor controller/actuator 420 may be present so as to rotate the lead screw 406, thus translating into linear motion of the moveable drive element 408. The motor 418 and actuator/controller 420 may rotate in either direction, thus driving the plunger 410 into the barrel 412 (ejecting fluid from the barrel chamber 422), or withdrawing (drawing medium into the barrel chamber 422).

Also shown in FIG. 4, there may be a mechanism for measuring the fluid ejected from the API 404. As depicted, a fixed potentiometer 424 with a movable wiper blade 426 attached to the moveable screw drive element 408 may assist in determining the volume of fluid ejected from the barrel chamber 422.

In another aspect, the hand-held input device 402 may be used to transmit signals S (as shown, wireless) to a receiver 428 associated with the API 404. The signal receiver 428 may send signal information to the processor 430 for processing the data. In turn, the processor 430 may send signals to a motor controller/actuator 420 to drive the motor 418, as is signaled by the hand-held input 402. Ultimately, the plunger 410 may be deployed to eject fluid from the barrel chamber 422 and to a conduit to a patient (e.g., via a needle, catheter, etc.—not shown).

FIG. 4 depicts a configuration wherein the signal receiver 428 and processor 430 may be housed in an API housing 432. However, it is contemplated that the signal receiver 428 and processor 430 may be a separate entity, and that the processed data/information could be transferred to the motor controller/actuator 420 by second transmitter/receiver (or more) between the processor 430 and the motor controller 420.

Furthermore, FIG. 4 depicts a measurement apparatus (e.g., potentiometer 424) that may be used to measure an amount of medium (and, rate of injection, for example) ejected by the API 404. It is also possible that this apparatus may not be required since the hand-held input device 402 may also be capable of providing the ejection measurement information/data.

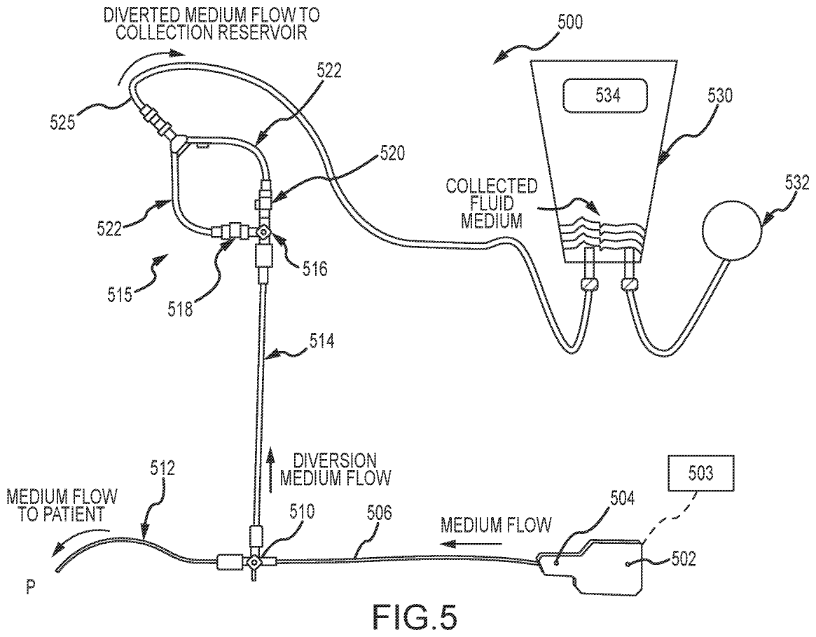

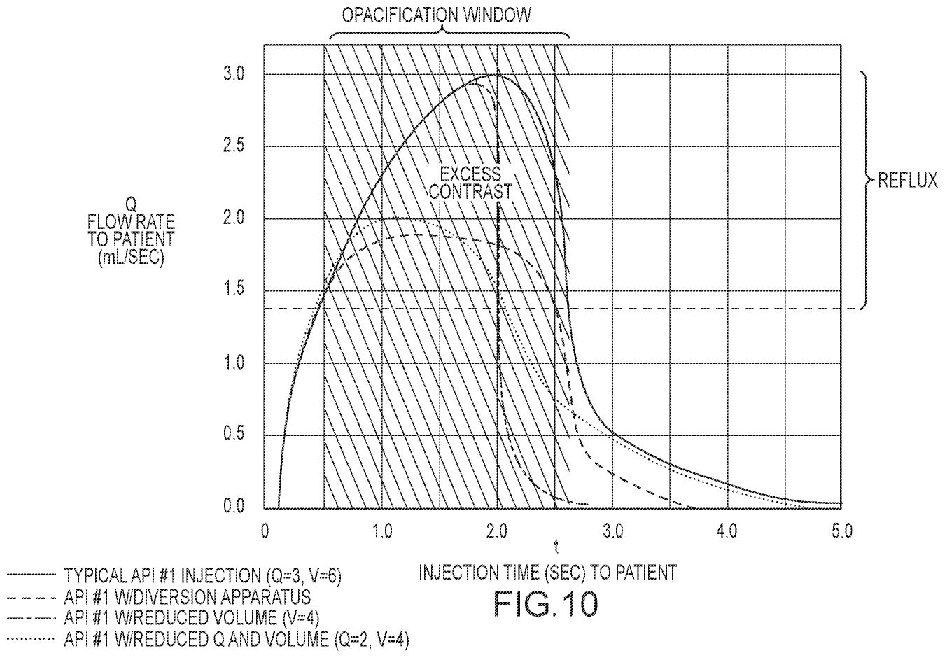

In the example of system 500 of FIG. 5, an API operator (such as a surgeon or other care provider) may select various baseline parameters of an API 502 (such as flow rate, injection volume, rise time, maximum injection pressure, etc.). These baseline parameters may be modified, or otherwise altered, by a diversion apparatus (such as an electro-mechanical pressure compensating valve, electro-mechanical diversion reservoir, etc.) to change an injection profile to mimic a more ac optimal injection profile (i.e., minimal contrast to achieve opacification). For example, from FIG. 10, the baseline parameters may provide a profile that may look like a “Typical API #1” injection. However, utilizing a diversion apparatus may modify the injection (e.g., in process, or real-time) to produce a profile similar to “API #1 w/diversion apparatus”, as illustrated.

FIG. 5 depicts a system 500 that may use an API 502 as described above, utilizing an input device 503 in the form of a hand-held input device. Thus, the system 500 depicted may inherently capture information/data as it relates to the medium ejected from, or introduced into, the chamber 504. In another aspect, the injector 502 may include a measurement apparatus as described in FIG. 4. The system 500 of FIG. 5, includes a diversion circuit 515 including a diversion conduit 514 utilized in modulating/altering the fluid medium to the patient P through a diversion valve 510 via a catheter 512. FIG. 5 identifies two diversion pathways 522, either of which may be selected based on the medium, location of injection to a patient site, and conduit used to deliver the medium (to name a few considerations). Each pathway 522 may be served by a valve 518, 520 allowing a different flow rate. FIG. 5 also depicts a collection reservoir 530 and measuring apparatus, such as a pressure gauge 532. The medium diverted away from the injection to the patient P may be collected in the reservoir 530. As depicted, the pressure gauge 532 may be capable of determining the volume of fluid medium collected in the reservoir 530 as a result of the “head pressure” of the fluid within the collection reservoir 530. The pressure gauge 532 and the collection reservoir 530 may be hung from a bag holder (not shown-such as an IV bag pole, or like). In another aspect, the collection reservoir 530 and the measuring/sensing device (i.e., pressure gauge 532) may be integrally constructed as a single device. Furthermore, whether integrally constructed or separate, information from the pressure gauge 532 may be directed to an output display 534, either by a wired or a wireless connection. In another example, the amount of diverted medium may be displayed on the display located on the API 502.

In determining the amount of medium injected to the patient P, the amount or volume of medium diverted may be subtracted from the total amount or volume of medium injected by the injector 502. To this end, a physician or system user may simply read the two values from output/data display 534 on the collection reservoir 530 and display on the API (such as depicted in FIG. 1) and may determine the amount injected to the patient P. Conversely, there may be a variety of ways to send the information to be processed to a remote device (such as via wired or wireless connections). The processor may be disposed in a separate component (e.g., an iPad) or could be combined with the injector 502, and/or a measuring sensor device.

In one embodiment, the data from the hand-held input device and the collection reservoir sensor (FIG. 5) may be delivered wirelessly (or via a wired connection) to a receiver and then a processor (FIG. 4). The data from these two data measurements may be used to calculate the volume of medium injected into the patient (e.g., total injected by the API 502 minus volume collected by the collection reservoir 530).

The diversion of medium from an injection, through the diversion valve 510, has been shown to be an advantageous modulator/controller of medium actually delivered to a patient P with an injection by hand, as well as by automated power injectors, such as API 502. The diversion valve 510 may provide for increasing resistance to a flow of medium into the diversion conduit 514 with increasing pressure of the medium being injected. That is to say, when there is little resistance to an injection from an injector (hand-held or API 502), a larger flow of the medium will be removed (through the diversion apparatus) out of the flow injected into the patient P. Conversely, if a much higher pressure is encountered in the conduits 506/514/512 from the injector 502 to the patient P, a lower amount of volume would flow through the diversion conduit 514. Generally, this type of modulation may allow for the actual injection into a patient P to rapidly attain a flow rate to the patient that is beneficial for evaluating a vessel or organ (for example), while buffering spikes of agent delivered to patient (e.g., flattening the curve of flow rate). Moreover, the diversion modulation may maintain a “duration” of an injection that may also be beneficial in the visualization (i.e., angiography) assessment. For example, in coronary angiography, if an injection into the coronary arteries (at a minimum flow rate) does not endure for about 3 or more heart beats, the duration of the injection may not be sufficient to actually visualize the artery well. Therefore, as an example, a person with an 80 beats per minute (0.75 seconds/beat), might need a minimum of 2.25 seconds (or more) to sufficiently evaluate the coronary artery.

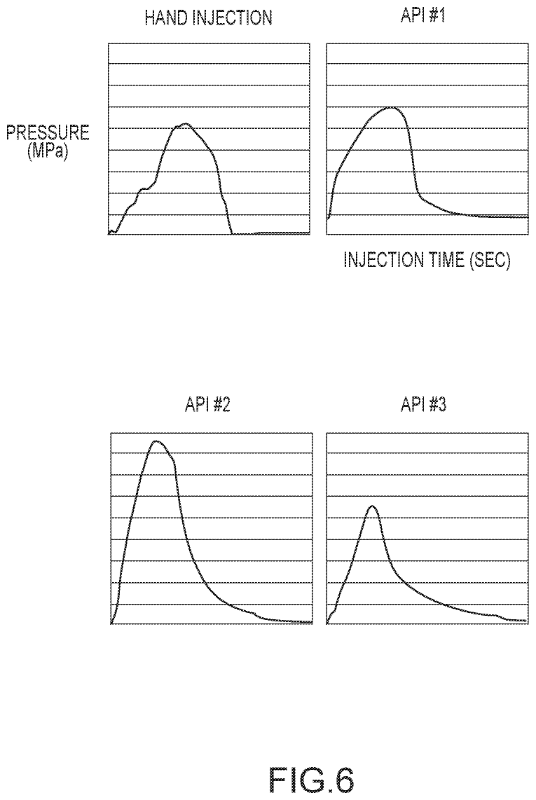

FIG. 6 presents some graphical results from an abstract titled: Comparison of Contrast Injection Pressure Contours with Different Methods for Coronary Angiography, SCAI 2020 Scientific Sessions, May 14-16, 2020, the disclosure of which is hereby incorporated by reference herein in its entirety, and which may be found at http://scai.confex.com/scai/2020/meetingapp.cgi/Paper/10225. As depicted, a hand-held syringe was used to inject a contrast medium, as well as three automated power injectors, API #1, API #2, and API #3. The performance testing included a digital pressure monitor being attached to a 3-way stopcock at the hub of a 4F angiographic catheter. The hand injection was derived during coronary angiography by a cardiologist. All API settings were standardized with a flow rate of 3 ml/sec, total volume of 6 ml, and rise of 0.5 second.

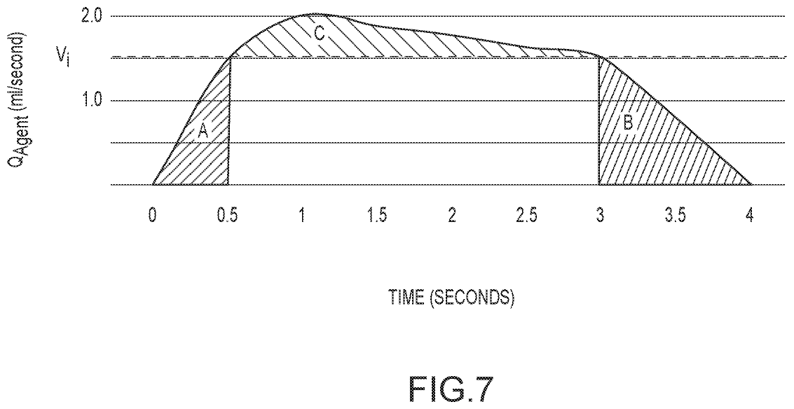

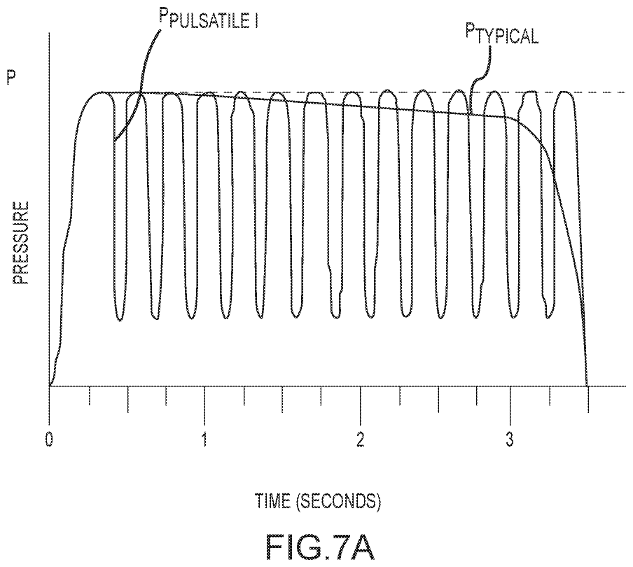

The data collected in FIG. 6 are presented as Pressure vs. Time. Although, the actual pressure contours are directly related to the flow rate (Q) since P=Q*R. R is the resistance, and in this case, the same catheter resistance R was tested in each scenario. Since R was equivalent, all of the pressure vs. time contours would be directly related to (same wave form) the flow vs. time profiles. Furthermore, the total volume (V=Q*T) injected into the catheter of each of the profiles is directly indicative of the area under each curve as shown. Of note in FIG. 6 is that all of the automated power injectors have some type of curve/flow profile. Indeed, the curve for each API results from each different API's ability to ramp quickly, back-off and stop quickly (e.g., the flow profile is not straight up, flattened Q, and straight down. Further, there is compliance in the tubing kits/manifolds, etc. that may influence the performance as well. Structurally, the motor needs to climb to a set flow rate/pressure, release pressure/flow rate at a set value, and quickly stop the injection. Thus, there may be systemic structural challenges in the system to actually provide such an injection profile (such as in FIG. 7) directly to the patient. As can be seen, each of the different API's have their own injection profile based at least in part on the construction of their injectors, including their piston, plunger and syringe barrel structures, for example.

An exemplary injection profile (Q vs. T) can be found in FIG. 7. In this case, an injection into a coronary left main may be anticipated. As depicted, a minimum flow rate Q is needed to observe contrast in the artery (Vi). This amount Vi is shown at approximately 1.5 ml/sec on FIG. 7, although this could be 1 ml/sec to 3 ml/sec, and preferably between 0.5 ml/sec and 6.0 ml/sec. Further, the flow rate required to be injected into the patient may vary given the vessel, or injection site. Referring to FIG. 7, various areas are shown which may have injection flows (Q) that are either insufficient to opacify the vessel appropriately (areas A and B), or are of a magnitude greater than is necessary for opacification, and may thus result in the over-delivering of contrast agent (area C). That is to say, if the injection had been controlled in the delivery of the contrast agent to obtain Vi (identified as a rectangle within the injection rate profile QAgent of FIG. 7), less contrast (25% to 30%) might have been used to achieve the same result (e.g., to sufficiently visualize the artery over the same period of time). In another example, only 50%-60% of the contrast attempted to be delivered to the patient would be needed to adequately visualize (i.e., opacify) the vessels. Other examples, e.g., about 10% to about 70%, about 20% to about 60%, or other ranges are also contemplated. Not only may it be important to have a minimum Q for opacification, depending on the injection and site, the duration of the injection may be important. If the duration (number of seconds at Vi) is too short, the operator may be unable to see what it is they are trying to assess. If the duration (number of seconds at Vi) is too long, more contrast may be used than necessary.

As a practical matter, and in further illustration of the complexity in efficiently delivering contrast agent into the dynamic environment of a coronary artery, some operators of the injector (a syringe, for example) may try to mimic a rapid injection so as to minimize the area of A in FIG. 7 through a rapid increase of pressure (and commensurate volume flow rate) with an injection. When sufficient opacification is “seen” radiographically, the operator may then decrease the pressure (and volume flow rate) of the injection. This technique may be helpful in reducing the area of A (quickly reaching Vi); however, the operator may “over-shoot” the delivery rate required for opacification (i.e., Vi) and thus increase the amount of over-injection which may be seen by area C in FIG. 7. It should be noted that a 10 cc (ml) syringe may be capable of injecting at 100 psi or more. This pressure of injection from the syringe could generate flows as high as, for example, 4.0 ml/second, which could be greater than what may be required to visualize the vessel.

Referring back to FIG. 6, the inventors have experienced on average up to 40% or more reduction of injection volume with hand-held syringes, while not jeopardizing visualization of the vessel through the use of a diversion apparatus to modulate/alter the injection into a patient. In certain examples, however, per injection reductions can vary from about 15% to about 60%, depending, for example, on the speed and pressure of the injection. The results achieved may be as described by FIG. 7. One of the benefits promoted by automated power injector manufacturers is the ability to better control the delivery of contrast injected into a patient, and thus reduce the amount of contrast injected. This may be partially true; however it is clear from FIG. 6 that the three API's tested revealed injection profiles that were not nearly the optimized curve as seen in FIG. 7.

FIG. 7A illustrates an exemplary pulsatile medium injection profile P_{Pulsatile I}, again over a typical 3.5 second injection of medium into a patient. The medium injection profile P_{Pulsatile I} of FIG. 7A may allow the attainment of full pressure of a typical injection to be realized, but do so at spaced intervals of medium pressurization. In the example illustrated in FIG. 7A, the “duty cycle” (time between waves) for the pulsatile pressure profile P_{Pulsatile I} may be about 0.25 seconds.

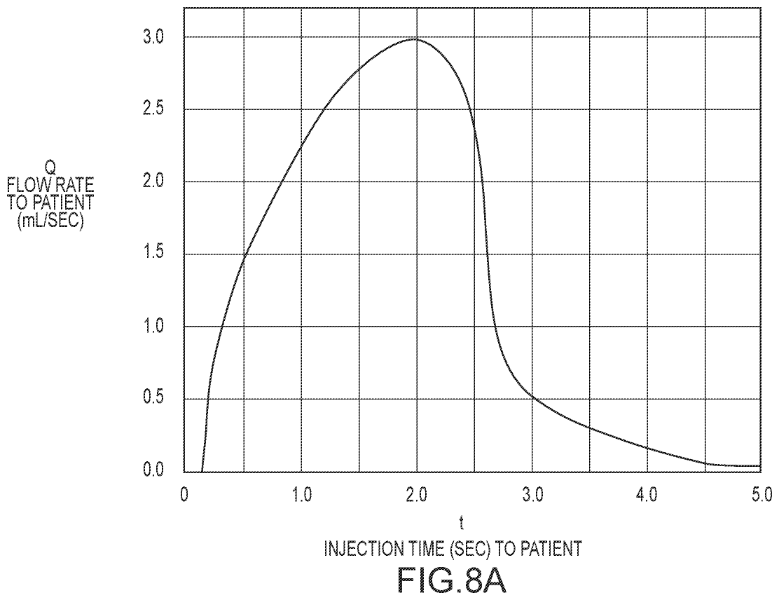

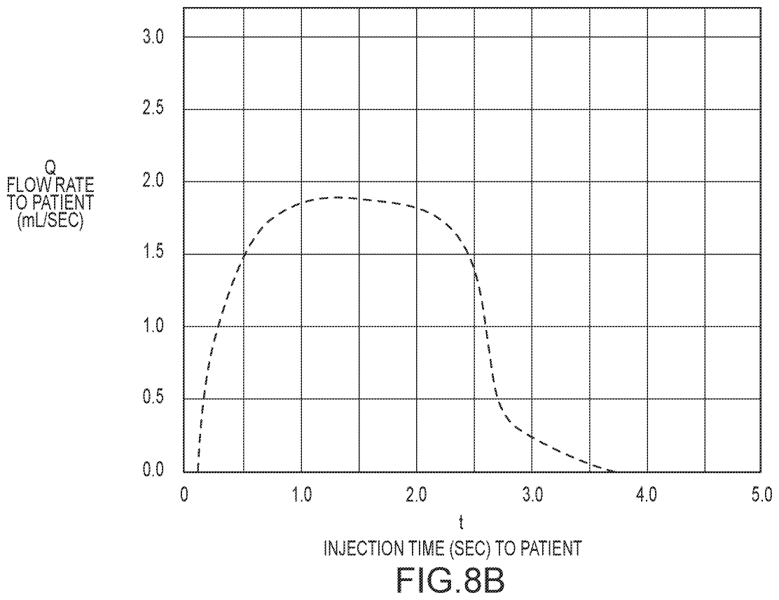

Referring to FIG. 8A, an injection profile of flow rate Q versus time is depicted, as derived from API #1 with a typical injector setting (Q=3, Total Volume injected=6 ml). The total volume used is the area under the curve. This is the injection profile actually delivered directly to the patient.

FIG. 8B depicts an injection profile (Q versus T) delivered directly to the patient if API #1 (FIG. 8A) utilized a diversion apparatus (as described by FIG. 5) to modulate/alter the injection from the injector with the same injector settings of FIG. 8A (Q=3, Total Volume=6 ml).

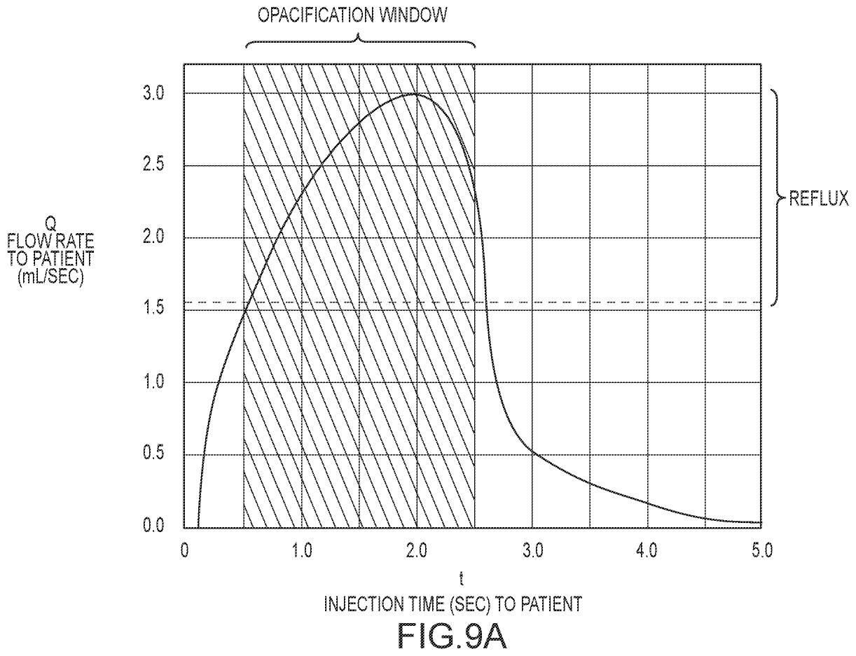

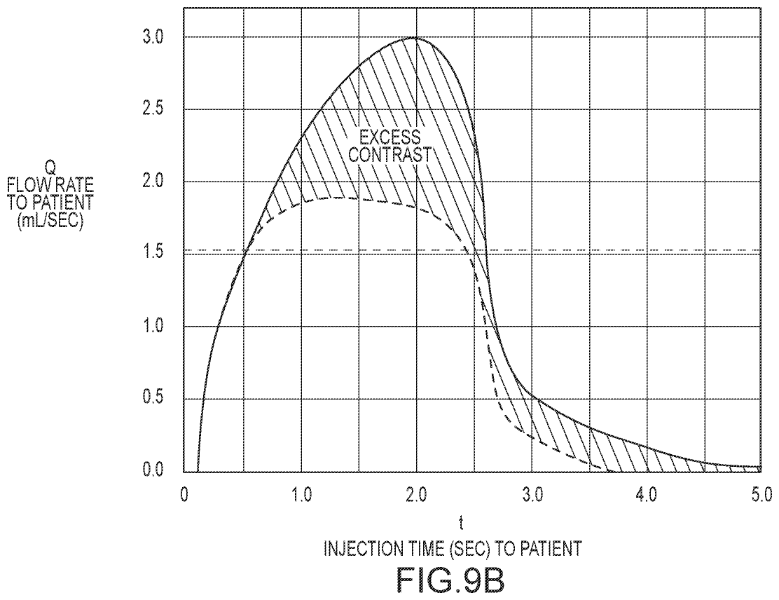

Depicted in FIG. 9A, a typical injection profile output (Q vs. t) from API #1 as shown in FIG. 8A, and including an “opacification window” superimposed on the injection profile. Depending on the application, certain flow rates may be required or desired to visualize the vessels. Rates of about 0.5 to about 3.0 ml/sec, about 1.0 to about 2.0 ml/sec, and about 1.25 to about 1.75 ml/sec are contemplated, as are flow rates of about 1.3, about 1.5, and about 1.6 ml/sec. A particular minimum flow rate may be needed to visualize the vessel, as well as at least 2 seconds duration over the selected flow rate, to the patient (Vi and duration described previously with respect to FIG. 7). This window is shaded in FIG. 9A, and as described previously, a flow rate below threshold (Vi) may result in streaming (i.e., wispy, inadequate opacification), while a flow rate above this threshold may result in reflux of contrast from the target (in this example, the coronary left main artery) artery into the aortic root.

Referring to FIG. 9B, the injection profiles of FIG. 8A (API #1) and FIG. 8B (API #1 utilizing a diversion apparatus) are superimposed on one another. Further highlighted in this example is the area (in grey) wherein the API injection profile curve is greater than the API #1 with the diversion apparatus curve. In essence, the API #1 (alone) delivers excess contrast (i.e., reflux), but still retains the flow rate, volume, duration that may be necessary to achieve adequate image quality (i.e. greater than Vi). This excess reflux may not be needed for radiographic imaging and may cause an additional, undesired & unnecessary contrast load on a patient's kidneys. As depicted, a desired opacification window of, for example, 2 seconds duration is shown. Further noteworthy in FIG. 9B is the combination (API #1 with the diversion apparatus of FIG. 5) may have the additional benefit of reducing the amount of contrast injected during the trailing, lower injection flow rate phase of the injection from approximately 2.5 seconds to 4.5 seconds. This contrast volume provides no added imaging benefit.

There may be other benefits associated with the use of a diversion apparatus in combination with an automated power injector. These benefits may include, for example, a smaller peak pressure (or flow rate) and a more constant (or flattened) profile over the duration.

FIG. 10 depicts changing the injection input on the API and the resulting injection profile directed to the patient. One curve is shown with the typical injection of the API #1 (Q=3 ml/sec, V=6 ml). Secondly, and over-layed, is the API #1 with a diversion apparatus (similar to FIG. 8B). In addition to these two curves, one profile depicts what the flow profile (Q versus t) with a smaller total volume of the API #1 injection, keeping the flow rate the same (Q=3 ml/sec). As may be seen, there is still excessive contrast injected to the patient and there is insufficient duration to provide opacification of the vessel. Furthermore, reducing the flow rate further (Q=2 ml/sec), with the total volume of 4 ml, may reduce some of the excess contrast injected into the patient, but may not provide sufficient medium due to the duration of the injection. As discussed previously, visualization of left main artery may require 2 to 4 beats, or more, of the heart pumping.

In addition to the systems described herein that utilize an API in conjunction with a diversion reservoir, the proposed technology contemplates duplicating the injection profile by programming an algorithm to mimic the effect created mechanically by the reservoir, while eliminating the diversion reservoir from the system. Such examples contemplate including a particular algorithm to control operation of the API, so as to mimic the effect of a diversion reservoir. Such a system may include the devices described herein, and their associated communications and processor/controller. Functionality of the algorithm may be programmed so the API includes a profile more similar to that depicted in FIG. 8B. As an example, such an algorithm may include generating an increase pressure prior to opening the syringe barrel outlet. A desired flow rate may be quickly or instantaneously established after opening the barrel outlet. Such flow may be maintained until a set flow duration has been established. The algorithm may further control the operation of the motor, e.g., to reverse operation of the motor (to generate a slight reverse flow) so as to quickly reduce the pressure (by reversing the flow rate). Other examples of algorithms that may mimic the functionality of a system that includes a diversion reservoir are contemplated. For example, the algorithm and appropriate components may be used to actuate one or more valves that affect fluid flow from the API, for example to relieve pressure, divert flow, and so on.

In an additional example, the system 400 of FIG. 4 and system 500 of FIG. 5 may include feedback loops to modify, or otherwise augment, manually selected baseline parameters of an API (e.g., flow rate, injection volume, rise time, maximum injection pressure, etc.). In these examples, these selected baseline parameters may be modified, altered, or otherwise augmented by real time feedback from various sensors (such as, potentiometer 424, flow/pressure transducer associated with API barrel 504/422, pressure/flow sensors associated with the injection into the patient, etc.), as well as any input device (such as, hand held device 402), to change the injection profile to match a predefined optimal injection profile (i.e., minimal contrast to achieve opacification). For example, from FIG. 10, baseline parameters may provide a profile that may look like a “Typical API #1” injection. However, sensors and input devices feedback may modify the injection in process, and in real-time, to provide an injection profile delivered to the patient more similar to what is achieved by “API #1 w/diversion apparatus”.

As described previously, the objective of obtaining optimal image opacity utilizing an API may be attempted by pre-setting a combination of specific API variables (e.g., contrast injection flowrate, volume, rise-time and/or injection pressure, as examples). As also discussed previously, an API operator may also be trying to minimize the contrast load/dose to a patient. An operator may rely on pre-selected settings on the API, or the user may need to adjust these settings prior to an injection and/or titrate the flowrate from the API real-time by using a variable rate hand controller, thus relying on the operator's interpretation of the fluoroscopic/X-Ray image to further guide API settings and/or titrate the flowrate with a hand controller or input device. In this case, the API operator may depend on their real-time image/opacification assessment, as well as incur an associated reaction time in the ability to achieve optimized opacification with minimized contrast dose.

Other examples that may allow for optimal image opacification while reducing the amount of contrast injected into the patient are contemplated. For example, each API injection may utilize feedback from a signal (data/information) derived from a fluoroscopic/X-Ray image to directly (or, indirectly) control, adjust or otherwise provide input, to the API drive mechanism (e.g., motor controller/actuator 420), without relying solely on the operator input. Other inputs are also contemplated, such as an EKG (pacing the heart beats), a pressure gauge associated with a guide catheter, a flow wire, etc. wherein the injection may be paced to the filling of the coronary arteries, for example, that may also be used to provide feedback so as to control, alter, or otherwise provide input to the injector. In this case, as exemplified, a fluoroscopic image is described; however, other inputs may be used and the fluoroscopic image is only one example.

Further, an opacification of a fluoroscopic/X-Ray image may be assessed via software to assist in determining, in real-time, if the opacity on an image might need to be more, or less, opacified. Processed data/information may be transferred to, for example, the motor controller/actuator 420. The processed data may be performed through one or more transmitters/receivers between the processor 430 and the motor controller 420. This data may be utilized to automatically adjust the API injection flowrate profile to arrive at, and maintain, the desired opacification for a set length of time. As described previously, the length of time for opacification could be quantified in terms of patient's number of heart beats per second. Furthermore, an API injection may terminate after a desired opacification is reached, and held, for the desired “opacification window”. It is further contemplated that operators/users may have individual preferences in opacification of a site. In this case, the operator/user (or another individual) may rate the “opacification” (e.g., higher or lower opacity and/or shorter or longer opacification duration). This rating could be performed post-injection, as well as it could be utilized to adjust future injections from the API. Moreover, this information may assist in the API (or other associated data processing system) to learn (i.e., artificial intelligence) operators' preferences in performing future injections, as well as specific opacification requirements (such as, site location, patient size, heart rate, to name a few). As an example, prior to an injection, a user could digitize the location of therapeutic interest (i.e., left main coronary artery, below the knee vessel, specific location within a vessel or organ, entire left coronary artery coronary tree, right coronary artery, PAD runoff, to name a few). In addition, one might use the data, and/or ratings, in assessing aortic reflux in images so as to reduce/diminish excess medium from being injected into a patient with the API providing better (e.g., efficient) injection profile.

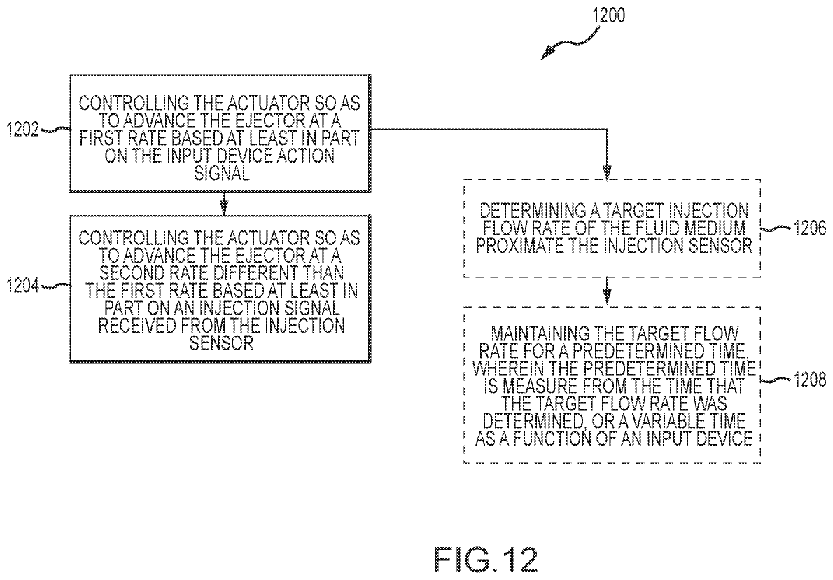

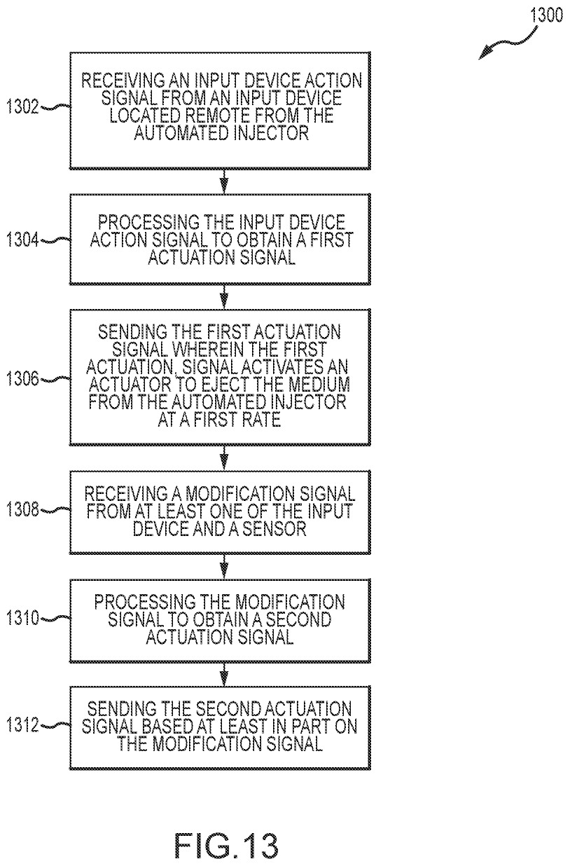

There are a variety of uses of an API and a variety of agents that may be injected to multiple injection sites within a patient. For this reason, the examples provided above, including the various flow rates, pressures, times of system operation, etc., are for illustrative purposes only, and actual values may change (even significantly) between various injectors and uses. Examples of systems operating with an algorithm are depicted in the context of FIGS. 12 and 13.

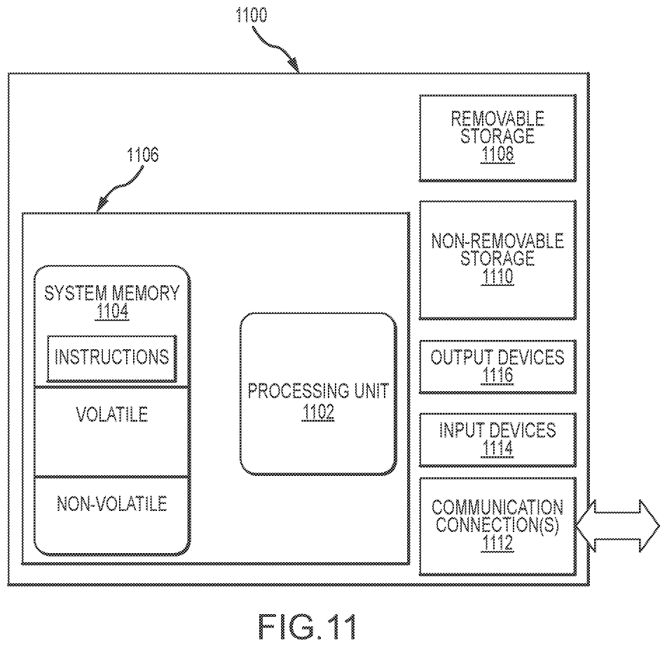

FIG. 11 illustrates one example of a suitable operating environment 1100 in which one or more of the present embodiments may be implemented. This is only one example of a suitable operating environment and is not intended to suggest any limitation as to the scope of use or functionality. Other well-known computing systems, environments, and/or configurations that may be suitable for use include, but are not limited to, personal computers, server computers, hand-held or laptop devices, multiprocessor systems, microprocessor-based systems, programmable consumer electronics such as smart phones, network PCs, minicomputers, mainframe computers, smartphones, tablets, distributed computing environments that include any of the above systems or devices, and the like.

In its most basic configuration, operating environment 1100 typically includes at least one processing unit 1102 and memory 1104, e.g., which may be contained in the API, the hand-held syringe, or another device remote from both. Depending on the exact configuration and type of computing device, memory 1104 (storing, among other things, instructions to perform the methods described herein) may be volatile (such as RAM), non-volatile (such as ROM, flash memory, etc.), or some combination of the two. This most basic configuration is illustrated in FIG. 11 by line 1106. Further, environment 1100 may also include storage devices (removable, 1108, and/or non-removable, 1110) including, but not limited to, magnetic or optical disks or tape. Similarly, environment 1100 may also have input device(s) 1114 such as touch screens, keyboard, mouse, pen, voice input, etc. and/or output device(s) 1116 such as a display, speakers, printer, etc. Also included in the environment may be one or more communication connections, 1112, such as LAN, WAN, point to point, Bluetooth, RF, etc.

Operating environment 1100 typically includes at least some form of computer readable media. Computer readable media can be any available media that can be accessed by processing unit 1102 or other devices comprising the operating environment. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, RAM, ROM, EEPROM, flash memory or other memory technology, CD-ROM, digital versatile disks (DVD) or other optical storage, magnetic cassettes, magnetic tape, magnetic disk storage or other magnetic storage devices, solid state storage, or any other tangible medium which can be used to store the desired information. Communication media embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The operating environment 1100 may be a single computer operating in a networked environment using logical connections to one or more remote computers. The remote computer may be a personal computer, a server, a router, a network PC, a peer device or other common network node, and typically includes many or all of the elements described above as well as others not so mentioned. The logical connections may include any method supported by available communications media. Such networking environments are commonplace in offices, enterprise-wide computer networks, intranets and the Internet. In some embodiments, the components described herein comprise such modules or instructions executable by computer system 1100 that may be stored on computer storage medium and other tangible mediums and transmitted in communication media. Computer storage media includes volatile and non-volatile, removable and non-removable media implemented in any method or technology for storage of information such as computer readable instructions, data structures, program modules, or other data. Combinations of any of the above should also be included within the scope of readable media. In some embodiments, computer system 1100 is part of a network that stores data in remote storage media for use by the computer system 1100.

FIG. 12 depicts a method 1200 of controlling ejection of a medium from an automated injector. The automated injector may be utilized in conjunction with an input device, such as the hand-held device depicted elsewhere herein. A processor may receive the signals from one or more sensors on the input device that indicate a position of the plunger of the input device within the housing thereof, as well as other aspects of the input device trigger (such as, for example, the speed at which the trigger/plunger is deployed/retracted, the pressure expressed on the trigger device). These signals may be further processed by the processor to actuate the automated injector accordingly. The processor may be located on the automated injector, input device, or may be multiple processors across multiple devices. In examples, locating the processor on the automated injector may be advantageous, since the automated injector is typically not disposable. Specific position sensors are described elsewhere herein. The method 1200 begins with operation 1202, controlling the actuator so as to advance the ejector at a first rate based at least in part on the input device action signal. Flow continues to operation 1204, controlling the actuator so as to advance the ejector at a second rate different than the first rate based at least in part on an injection signal received from an injection sensor. In examples, the injection sensor (and associated signal) may be for pressure, volumetric flow, flow rate, and so on). In examples, operation 1206 includes determining a target flow rate of the fluid medium proximate the injection sensor. This target flow rate may be determined based on the injection signal. Thereafter, operation 1208, may maintain a target flow rate for a predetermined time, wherein the predetermined time may be measured from the time that the target flow rate was determined or established. In other examples, the time may be based in part on the time associated with a number of beats of a human heart. In still other examples, time may be a variable time as a function of an input device, such as when an operator wants to inject short “puffs” of contrast in locating diagnostic and/or therapeutic equipment within the heart and/or its vessels. Further, it is anticipated that there may also be a combination of pre-set time interval and/or a variable interval. As an example, an injection interval may allow for short “puffs” if the input device is activated for less than a set amount of time (for example, 1 or 2 seconds). However, activating the input device more than this amount of time may trigger a pre-set injection interval. Furthermore, if the injection interval were signaled to be longer than a pre-set amount, the injector may continue as a variable input signal to allow continued injection by the API. These are just a few examples wherein the injection may be pre-determined and/or variable.

FIG. 13 depicts an exemplary method 1300 of controlling ejection of a medium from an automated injector. The method 1300 begins with operation 1302, receiving an input device action signal from an input device located remote (wired or wireless) from the automated injector. Thereafter, operation 1304, processing the input device action signal to obtain a first actuation signal, may be performed. The first actuation signal is sent in operation 1306; this signal activates an actuator to eject the medium from the automated injector at a first rate. Thereafter, a user of the input device may change the rate of advancement of the plunger, or a direction thereof (from advancing to retracting) for a number of reasons, such as requirements of the particular procedure, experience, etc. Such an action causes a modification signal to be received from at least one of the input device and a sensor, operation 1308. This modification signal is then processed to obtain a second actuation signal, operation 1310. This the second actuation signal is then sent in operation 1312.

This disclosure described some examples of the present technology with reference to the accompanying drawings, in which only some of the possible examples were shown. Other aspects can, however, be embodied in many different forms and should not be construed as limited to the examples set forth herein. Rather, these examples were provided so that this disclosure was thorough and complete and fully conveyed the scope of the possible examples to those skilled in the art.

Although specific examples were described herein, the scope of the technology is not limited to those specific examples. One skilled in the art will recognize other examples or improvements that are within the scope of the present technology. Therefore, the specific structure, acts, or media are disclosed only as illustrative examples. Examples according to the technology may also combine elements or components of those that are disclosed in general but not expressly exemplified in combination, unless otherwise stated herein. The scope of the technology is defined by the following claims and any equivalents therein.

What is claimed is:

1. A system for injecting medium into a patient, the system comprising:
an automated injector comprising:
a medium reservoir;
an ejector for ejecting a volume of a fluid medium from the medium reservoir; and
an actuator coupled to the ejector;
an input device remote from and communicatively coupled to the actuator, wherein the input device comprises:
a syringe housing;
a plunger slidably received in the syringe housing;
a circuit board coupled to a first component of the input device;
a plunger position sensor;
a battery coupled to the circuit board and configured to provide power to the plunger position sensor; and
a transmitter coupled to the circuit board for sending an input device action signal to the automated injector, wherein the input device action signal is based at least in part on a signal sent from plunger position sensor; and
a diversion apparatus disposed downstream from the reservoir, wherein the diversion apparatus is configured to receive at least a first portion of the volume of the fluid medium ejected from the medium reservoir.

2. The system of claim 1, wherein the transmitter comprises a wireless transmitter and wherein the automated injector comprises a wireless receiver for receiving the input device action signal.

3. The system of claim 1, wherein the input device further comprises a spring for biasing the plunger relative to the syringe housing.

4. The system of claim 1, wherein:
the medium reservoir comprises a syringe barrel;
the ejector comprises a plunger slidably disposed in the syringe barrel; and
the actuator comprises a lead screw and a motor coupled to the lead screw, wherein a rotation of the lead screw advances the ejector within the syringe barrel.

5. The system of claim 4, wherein the automated injector further comprises a position sensor for detecting a position of at least one of the ejector and the lead screw.

6. The system of claim 1, further comprising a patient connection element downstream of the diversion apparatus for receiving at least a second portion of the volume of the fluid medium ejected from the medium reservoir.

7. The system of claim 6, wherein the first portion of the volume of the fluid medium and the second portion of the volume of the fluid medium comprise the volume of the fluid medium ejected from the medium reservoir.

8. The system of claim 1, wherein the diversion apparatus comprises a waste vessel for receiving at least a portion of the first portion of the volume of the fluid medium.

9. The system of claim 1, wherein the plunger position sensor comprises at least one Hall Effect sensor coupled to the first component and a magnet coupled to a second component of the input device, wherein the first component is moveable relative to the second component.

10. The system of claim 1, wherein the plunger position sensor comprises at least one of a light emitter, a light receiver, a potentiometer, and a magnet.

11. A system for injecting medium into a patient, the system comprising:
an automated injector comprising:
a medium reservoir;
an ejector for ejecting a volume of a fluid medium from the medium reservoir; and
an actuator coupled to the ejector;
an injection sensor disposed proximate an outlet of the medium reservoir;
an input device remote from and communicatively coupled to the actuator, wherein the input device comprises:
a syringe housing;
a plunger slidably received in the syringe housing;
a circuit board coupled to a first component of the input device;
a plunger position sensor;
a battery coupled to the circuit board and configured to provide power to the plunger position sensor; and
a transmitter coupled to the circuit board for sending an input device action signal to the automated injector, wherein the input device action signal is based at least in part on a signal sent from plunger position sensor;
a processor; and
memory storing instructions that, when executed by the processor, cause the automated injector to perform operations comprising:
controlling the actuator so as to advance the ejector at a first rate based at least in part on the input device action signal; and
controlling the actuator so as to advance the ejector at a second rate different than the first rate based at least in part on an injection pressure signal received from the injection sensor.

12. The system of claim 11, wherein the processor and the memory are disposed on the automated injector.

13. The system of claim 11, wherein the position sensor comprises at least one of a Hall Effect sensor, light emitter, a light receiver, a potentiometer, and a magnet.

14. The system of claim 11, wherein controlling the actuator so as to advance the ejector at a second rate comprises:
determining a target flow rate of the fluid medium proximate the injection sensor; and
maintaining the target flow rate for a predetermined time, wherein the predetermined time is measured from the time that the target flow rate was determined or a variable time as a function of an input device.

15. The system of claim 14, wherein the target flow rate is determined based at least in part on the injection pressure signal sent from the injection sensor.

16. The system of claim 14, wherein the target flow rate is determined based at least in part on a signal sent from a flow sensor.

17. A method of controlling injection of a medium into a patient with an automated injector, the method comprising:
receiving an input device action signal from an input device located remote from the automated injector;
processing the input device action signal to obtain a first actuation signal;
sending the first actuation signal, wherein the first actuation signal activates an actuator to eject the medium from the automated injector at a first rate;
receiving a modification signal from at least one of the input device and a sensor;
processing the modification signal to obtain a second actuation signal; and
sending the second actuation signal based at least in part on the modification signal.

18. The method of claim 17, wherein the sensor is disposed remote from the automated injector.

19. The method of claim 18, wherein the sensor senses a pressure within a medium delivery system fluidically coupled to the automated injector and the patient.

20. The method of claim 16, wherein the sensor is disposed within the automated injector and senses a pressure of medium within a medium reservoir of the automated injector.
